(19) United States
(12) Patent Application Publication
(10) Pub. No.: US 2010/0140112 A1
(43) Pub. Date: Jun. 10, 2010

(54) Nanoparticle Marker, Diagnostic Methods Using the Same and Diagnostic Kit and Apparatus Using the Same

(76) Inventors: Kyoung-Sik Seo, Gwangju (KR); Jeong-Whan Kim, Gwangmyeong-si (KR)

Correspondence Address: CLARK & ELBING LLP, 101 FEDERAL STREET, BOSTON, MA, 02110, US

(21) Appl. No.: 11/993,715

(22) PCT Filed: Jun. 23, 2006
(86) PCT No.: PCT/KR2006/002440

§ 371 Date: Sep. 15, 2009

(30) Foreign Application Priority Data
Jun. 23, 2005 (KR) 10-2005-0054516
Jun. 12, 2006 (KR) 10-2006-0052726

(51) Int. Cl. G01N 27/26; C08F 290/14; C08G 63/91; C07F 1/08; C07F 1/00; C07F 3/00; C07F 7/24; C07F 3/06; C07F 3/08; C07F 15/04; G01N 33/553

(52) U.S. Cl. 205/780.5; 525/50; 525/54.2; 525/54.1; 556/110; 556/118; 556/81; 556/64; 556/138; 556/45; 204/403.01; 436/525; 205/792; 205/782; 205/787; 204/403.02

(57) ABSTRACT

A diagnostic kit disclosed herein comprises a nanoparticle-biomaterial complex, an extraction solution, a collection electrode, and a current peak measurement unit. The nanoparticle-biomaterial complex comprises: one or more nanoparticles selected from a metal group consisting of zinc, cadmium, lead, copper, gallium, arsenic, thallium, nickel, manganese and bismuth; one or more biomaterial-binding materials binding to the nanoparticles through a binding-stabilizing agent and binding specifically to the biomaterials to be detected; and a binding-stabilizing agent forming bonds between the nanoparticles and the biomaterial-binding materials. The extraction solution serves to isolate and extract the nanoparticles from the nanoparticle-biomaterial complex. The collection electrode serves to collect the nanoparticles from the extraction solution. The current peak measurement unit serves to measure current peaks corresponding to the nanoparticles collected from the collection electrode. A diagnostic kit disclosed herein comprises a nanoparticle-biomaterial complex, an extraction solution, a collection electrode and a current peak measurement unit. The nanoparticle-biomaterial complex comprises in the diagnostic kit: one or more nanoparticles selected from a metal group consisting of zinc, cadmium, lead, copper, gallium, arsenic, thallium, nickel, manganese and bismuth; one or more biomaterial-binding materials binding to the nanoparticles through a binding-stabilizing agent and binding specifically to the biomaterials to be detected; and a binding-stabilizing agent inducing the binding between the nanoparticles and the biomaterial-binding materials. The extraction solution serves to isolate and extract the nanoparticles from the nanoparticle-biomaterial complex. The collection electrode serves to collect the nanoparticles from the extraction solution. The current peak measurement unit serves to measure current peaks corresponding to the nanoparticles collected from the collection electrode. A diagnostic device disclosed herein is an information technology-integrated, miniaturized electrochemical biosensor, which comprises a disposable tip, an electrode, a container for storing a diagnostic reagent, and a unit for performing electric measurement or optical measurement, is in the form of a pipette or syringe, and has a container stopper in which the electrode is included.

### TECHNICAL FIELD

The present invention relates to a nanoparticle label, a diagnostic method and kit that use the same, and a diagnostic device that uses the same. More particularly, the present invention relates to an electrochemical diagnostic method and kit that use the oxidation/reduction ability of a nanoparticle marker, and a diagnostic device that uses the same.

### BACKGROUND ART

Recently, there have been attempts to diagnose various disease conditions using proteomic sensing systems. For this purpose, the proteomic sensing systems should ensure convenient operations for users and low cost and achieve excellent sensitivity, selectivity and reproducibility.

The proteomic sensing systems are mainly used as diagnostic systems, and typical examples thereof include immune sensors recognizing antigens or antibodies.

Such diagnostic systems should ensure a method capable of detecting a given biomaterial (protein or DNA etc) for diagnosis. As prior methods for detecting biomaterials, fluorescent labeling methods that use organic dyes or the like have been known. Fluorescent labels emit various colors depending on the kind thereof to provide means for detecting target biomaterials.

Meanwhile, when pluralities of biomaterials are to be simultaneously detected, pluralities of fluorescent labels that emit different colors are required. However, when a plurality of colors are simultaneously emitted as described above, a phenomenon, called “photobleaching”, can occur. Also, prior fluorescent labels have a problem in that they have narrow optical excitation and emission bands. In addition, when they bind to biomaterials, they can adversely affect the activity of the biomaterials.

For this reason, there is a need for a labeling method, which overcomes this problem with the prior labeling method and is physically more stable and functional. At the same time, a more stable and exact method of detecting a plurality of biomaterials is required.

Meanwhile, according to such requirements, labeling methods using semiconductor quantum dot (hereinafter, referred to as “QD”) nanoparticles have recently been known. The prior QD nanoparticles are physically stable compared to fluorescent labels, but have a low ability to bind to biomaterials to be labeled, and do not overcome limitations on the surface processing thereof. For this reason, the prior QD nanoparticles have been used only as a label source optical analysis.

Thus, there is a need for a labeling method which uses novel nanoparticles, which can successively bind to biomaterials and easily detect the biomaterials.

Recently, biological analysis systems have been continuously developed according to the requirement of self-diagnosis for human genome project studies and medical purposes, because they have an advantage in that they can perform the analysis of biological samples in a rapid, convenient and cost-effective manner. However, prior biosensors and biochips seemed to be promising, but currently encounter an ultimate limitation in terms of practical use. Also, laboratory-scale biosensor systems still have a lot of technical limitations, and stable analytic systems for industrial applications are still insufficient. Particularly, proteomic sensing systems have an advantage in that the application thereof can selectively widen for complicated functions in different cell types and the diagnosis of various disease conditions. However, there is a need to realize a diagnostic system, which makes prior laboratory-scale protein diagnosis more substantial, is convenient to use, have functions equal to those of large-scale study systems, and have excellent sensitivity, selectivity and reproducibility.

Immunoassay systems are protein analytic methods providing users with high fidelity, and are the most reliable means for early diagnosing human diseases, such as kidney diseases, diabetes, heart diseases and hypertension, in clinical applications. Typical examples thereof are immunoassay sensors, which can perform a wide, rapid, convenient and efficient immunological assay for not only the early diagnosis of patient diseases, but also the screening of various protein complexes. In addition, most studies have been concentrated on multi-analyte immunoassays with multicolor fluorescence analysis. However, such optical-based immunoassays generally employ organic dye fluorescent labels, and thus are confronted with a lot of limitations, even though they have high optical sensitivity as described above. Also, the fluorescent dyes have a problem in that they react with biomolecule surfaces to cause damage to the biological functionality of the biomolecules.

To overcome these problems occurring in the optical analysis systems, electrochemical sensing technology has been developed and attempted, which have advantages in that they use a simple process, require low cost and make miniaturization easy, compared to optical assays. However, the development thereof have progressed over a few years, the introduction of commercialized systems in the biomedical field is still very insufficient.

Furthermore, systems for diagnosing blood glucose and complications thereof, which are currently used in hospitals and medical-related agencies, utilize a complicated reagent treatment process and waste much time. In addition, interfaces limited to complicated and expensive equipment and experts actually make it very difficult for inexpert patients to perform early diagnosis.

### DISCLOSURE

### Technical Problem

Accordingly, the present invention has been made to solve the above-described problems occurring in the prior art, and it is an object of the present invention to provide a novel nanoparticle label, which has an excellent ability to bind to biomaterials, has high purity and is physically stable.

Another object of the present invention is to provide a novel diagnostic kit including a novel nanoparticle label, which has an excellent ability to bind to biomaterials and is physically and chemically stable.

Still another object of the present invention is to provide a diagnostic method that uses a novel nanoparticle label, which has an excellent ability to bind to biomaterials and is physically and chemically stable.

Yet still another object of the present invention is to provide a diagnostic device, which enables point-of-care testing to be immediately performed using a nanoparticle label.

### Technical Solution

According to one aspect of the present invention for achieving the above-described objects, there is provided a nanoparticle-biomaterial complex comprising: one or more nanoparticles selected from a metal group consisting of zinc, cadmium, lead, copper, gallium, arsenic, thallium, nickel, manganese and bismuth; a specific biomaterial; and a binding-stabilizing agent containing a polymer chain, which has at one side thereof a substituent group, having a charge property capable of binding the stabilizing agent to the nanoparticle, and has a plurality of water-soluble substituent groups at the opposite side, whereby the binding-stabilizing agent binds to the nanoparticle through the substituent group at the one side, stabilizes the nanoparticle through the water-soluble substituent groups at the opposite side, and forms bonds with the biomaterials through the water-soluble groups.

According to another aspect of the present invention, there is provided a method for preparing nanoparticles, the method comprising the steps of: allowing hexadecanol, potassium hydroxide and carbon disulfide to react with each other to prepare a hexadecyl xanthate (hereinafter, referred to as “HDX”) potassium salt; allowing the obtained HDX potassium salt to react with one or more nanoparticles selected from a metal group consisting of zinc, cadmium, lead, copper, gallium, arsenic, thallium, nickel, manganese and bismuth, so as to prepare HDX metal sulfide nanoparticles; and allowing the HDX metal sulfide nanoparticles to react with a specific alkylamine dopant to prepare metal sulfide nanoparticles.

According to still another aspect of the present invention, there is provided a diagnostic kit comprising a nanoparticle-biomaterial complex, an extraction solution, a collector electrode and a current peak measurement unit. The nanoparticle-biomaterial complex comprises: one or more nanoparticles selected from a metal group consisting of zinc, cadmium, lead, copper, gallium, arsenic, thallium, nickel, manganese and bismuth; one or more biomaterial-binding materials, which bind to the nanoparticles through a binding-stabilizing agent and bind specifically to biomaterials to be detected; and a binding-stabilizing agent forming bonds between the nanoparticles and the biomaterial-binding materials. The extraction solution serves to isolate and extract the nanoparticles from the nanoparticle-biomaterial complex. The collection electrode serves to collect the nanoparticles from the extraction solution. The current peak measurement unit serves to measure current peaks corresponding to the nanoparticles collected from the collection electrode.

According to still another aspect of the present invention, there is provide a diagnostic method comprising the steps of: determining one or more biomaterial-binding materials which can bind specifically to one or more biomaterials to be detected; selecting one or more nanoparticles from the group consisting of zinc, cadmium, lead, copper, gallium, arsenic, thallium, nickel, manganese and bismuth, and binding the selected nanoparticles to the biomaterial-binding materials to form one or more nanoparticle-biomaterial complexes; placing the nanoparticle-biomaterial complexes in a sample to be diagnosed, and mixing the complexes with the sample to induce the binding between the biomaterials to be detected and the nanoparticle-biomaterial complexes; isolating the nanoparticle-biomaterial complexes bound specifically to the biomaterials; separating and collecting a nanoparticle from the isolated nanoparticle-biomaterial complexes; and measuring a characteristic current peak corresponding to the collected nanoparticles.

According to still another aspect of the present invention, there are provided: a dopette-type diagnostic device 400 which is connected to a rack-type docking container 500 comprising an external potentiostate; a micropipette-type diagnostic device comprising a disposable tip 300 and a body 200; and a stopper-type diagnostic device, which is connected with a potentiostat through a stopper 110 including a triode electrodes, and a connection 40.

Hereinafter, embodiments of the present invention will be described in detail with reference to the accompanying drawings such that they can be readily conceived by those skilled in the art. It is to be understood, however, that the present invention can be embodied in various different forms and are not limited to the embodiments disclosed herein.

The present invention provides a novel nanoparticle label which can be used as a signaling label for biomaterials.

As used herein, the term “biomaterials” refers to materials present in organisms, which, when detected with a specific label, can be used for biological applications. Specifically, the biomaterials include nucleic acid such as DNA or RNA, amino acid, nucleic acid-amino acid complexes, or antibodies.

In the embodiment of the present invention, examples of the biomaterial include kidney and heart disease-related early clinical markers, human serum albumin (HSA), human β₂-microglobulin (MG), human myoglobin (Mb), C-reactive protein (CRP) and the like.

According to the method of detecting the nanoparticle according to the embodiment of the present invention, the concentration of the biomaterial as described above can be precisely measured. Meanwhile, when the method of detecting the nanoparticles according to the inventive embodiment is used, the user of the inventive detection method can warn a detection subject against infection with a specific disease according to whether the measured concentration of the biomaterial exceeds a specific concentration, or can use the measured concentration as a basis for the diagnosis of a specific disease. Specifically, it should be understood that a specific biomaterial associated with a specific disease such as diabetes or hypertension can be detected using the inventive nanoparticle as a label, and this detection progress can be used as a basis for the diagnosis of the relevant specific disease.

In the inventive embodiment, the nanoparticle used as the nanoparticle label is a metal-based nanoparticle having excellent resolution and signal selectivity. The nanoparticle used in the inventive embodiment is not specifically limited as long as it is a metal having excellent resolution and signal selectivity. As used in the inventive embodiment, the term “resolution” means that the peak width of a signal generated from the relevant metal is narrow so that it is distinguished from the peak of other signals without overlapping, and the term “signal selectivity” means an extent to which a signal peak produced from the relevant metal is easily distinguished from signal peaks produced from other metals. In other words, an increase in resolution leads to an increase in signal selectivity.

The metal nanoparticle according to the inventive embodiment is metal sulfide (hereinafter, referred to as “MS”) obtained according to a nanocrystal synthesis method to be described below. As a metal dissolved in the metal sulfide, zinc (Zn), cadmium (Cd), lead (Pb), copper (Cu), gallium (Ga), arsenic (As), thallium (Tl), nickel (Ni), manganese (Mn) or bismuth (Bi) is preferably used in the inventive embodiment. Particularly, zinc, cadmium, lead or copper is preferably used, because it produces a selective signal having more excellent resolution.

The size of this metal nanoparticle according to the inventive embodiment is similar to the size range of most biomaterials, and thus the metal nanoparticle easily forms a “nanoparticle-biomaterial complex” with the biomaterial.

The nanoparticle label according to the inventive embodiment can stably form a covalent bond with a biomaterial. As the biomaterial, a specific antibody detecting a causative protein indicative of a symptom of human disease is used. Then, the relevant nanoparticle is detected using the electrochemical characteristic of the metal nanoparticle label.

Accordingly, when the nanoparticle label according to the inventive embodiment is used, the received signal of the relevant nanoparticle label can be analyzed through an electrochemical assay to sense the presence of the specific antibody bound to the nanoparticle, thus detect the causative protein. According to the results of this analysis, the causative protein can be sensed, and according to the sensed results, a symptom of a specific disease can be diagnosed.

In another aspect, the present invention provides: a droppette-type diagnostic system 400 which is connected to a rack-type docking container 500 containing an external potentiostat; a micropipette-type diagnostic system comprising a disposable tip 300 and a body; and a stopper-type diagnostic system which is connected with a potentiostat through a stopper 110 including a triode electrode, and a connection 400. First, the dropette-type diagnostic system 400 comprises a suction device 10 for the suction of a biological sample, a sample inlet 20, a connection 40 to a rack-type docking container 500 including a potentiostat, and a triode electrode 30. The disposable dropette 400 can comprise a microporous membrane 15 for removing impurities from a biological sample. Then, the micropipette-type diagnostic system comprises a disposable tip 300 including a sample inlet 20 and a triode electrode 30, and a potentiostat-containing body 200, which includes a pipette module 11 capable of including various parts such as springs and gears, a connection 40 to the disposable tip 300, a mobile circuit 90 and a display module 100. The disposable tip 300 may comprise a microporous membrane for removing impurities from a sample. Also, regarding the stopper-type diagnostic system, a triode electrode 30 is inserted into a stopper 110 of a container and protruded into the container such that this electrode can come into contact with a biological sample portion. A signal measured through the triode electrode is transmitted to the external potentiostat through the connection 40.

### ADVANTAGEOUS EFFECTS

The kit for detecting and diagnosing a biomaterial using the inventive nanoparticle label can analyze the characteristic current peak of each metal nanoparticle, and thus can measure the biomaterial to be detected, in a convenient, quantitative and precise manner. Accordingly, the diagnostic kit that uses the inventive nanoparticle can show the results of detection and diagnosis in a rapid and convenient manner.

Also, the kit for detecting and diagnosing a biomaterial using the inventive nanoparticle label has a very low detection limit, such that it can precisely measure even a biomaterial (antigen or DNA) contained in a trace amount of a patient's sample (urine, blood or body fluid. Thus, the kit can be miniaturized.

Accordingly, the diagnostic kit that uses the nanoparticle as a label according to the inventive embodiment can electrochemically diagnose human diseases (diabetes, kidney diseases, heart diseases, etc.) in a rapid and convenient manner.

The inventive diagnostic device is in the form of a micropipette which is an information technology-integrated and miniaturized measurement device, having electrodes included in a container stopper. This diagnostic device can very precisely and conveniently diagnose a relevant biomaterial from very small amounts of patient's samples, including urine, blood and body fluid, and can easily perform point-of-care testing so as to help patients themselves to cope with various chronic diseases.

### DESCRIPTION OF DRAWINGS

FIG. 1a is a conceptual diagram schematically showing a method for detecting DNA using a nanoparticle label according to an embodiment of the present invention.

FIG. 1b is a conceptual diagram schematically showing a method for detecting an antigen using the nanoparticle label according to the inventive embodiment.

FIG. 2 is a conceptual diagram schematically showing a method for preparing a nanoparticle according to an embodiment of the present invention.

FIG. 3a is a conceptual diagram showing a method for preparing a nanoparticle-antibody complex according to an embodiment of the present invention.

FIG. 3b is a conceptual diagram showing a method for preparing a nanoparticle-DNA complex according to an embodiment of the present invention.

FIG. 4a is a graphic diagram showing barcodes obtained by dissolving ZnS-anti-β₂-MG, CdS-anti-Mb, PbS-anti-HSA and CuS-anti-CRP according to embodiments of the present invention in nitric acid and converting the resulting current peaks and the corresponding current peak signals into digital signals.

FIG. 4b is a graphic diagram showing a current peak signal in the case where there is no antigen in a sample to be detected.

FIGS. 4c to 4f are graphic diagrams showing barcodes obtained by converting current signals and the corresponding peak signals in digital signals, in the cases where there is one antigen target in each of samples to be detected.

FIG. 4g is a graphic diagram showing barcodes obtained by converting current peak signals and the corresponding current peak signals in the case where there are four kinds of antigen targets in a sample to be detected.

FIG. 5 is a sequence diagram showing a diagnostic method that uses a nanoparticle label according to an embodiment of the present invention.

FIG. 6 is a schematic view illustrating the main elements of (a) a disposable dropette-type diagnostic device and a micropipette-type diagnostic device including a disposable tip.

FIG. 7 is a model showing the substantial construction of a disposable dropette.

FIG. 8 is a schematic diagram illustrating an electrical analytical device in a docking-type diagnostic device, to which a pipette tip and a reagent container are connected. FIG. 8, (a) shows a perspective view of a rack-type docking container which is connected to a disposable dropette, and (b) shows a cross-sectional view of the container shown in (a).

FIG. 9 shows an immunoassay procedure for immunoassaying a patient's urine sample (urine protein) using the inventive diagnostic device.

FIG. 10 is a schematic diagram showing the simplest self-bioanalytic system, which comprises a disposable container and a stopper having an electrode attached thereto.

FIG. 11 is a graphic diagram showing negative electrode immuno-stripping current signals as functions of increases in the concentrations of three antigen substances, which were simultaneously analyzed using the inventive pipette-type sensor.

FIG. 12 is a schematic diagram showing various biosensor models obtained by applying the inventive diagnostic device.

### DESCRIPTION OF REFERENCE NUMERALS

10: Suction device; 11: general pipette module which can include various parts such as springs and gears; 15: microporous membrane; 20: sample inlet; 30: triode electrode; 40: connection to docking container; 50: display; 60: reaction chamber; 70: rack holder; 80: rare earth metal magnet; 90: mobile circuit; 100: small-scale strip sensor/display module; 110: stopper having an electrode attached thereto; 120; glass container for general testing; 130: cylindrical PVC platform; 200: body; 300: disposable tip; 400: dropette; and 500: rack-type docking container.

### BEST MODE

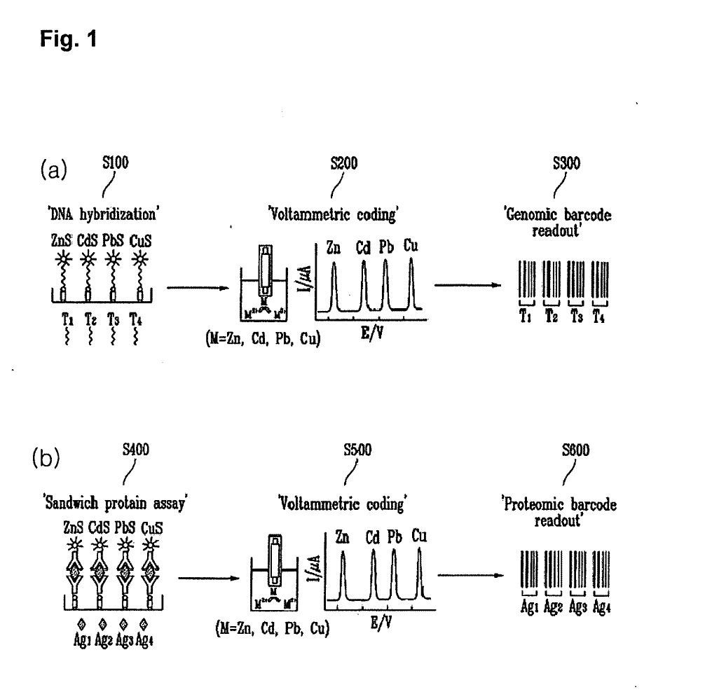

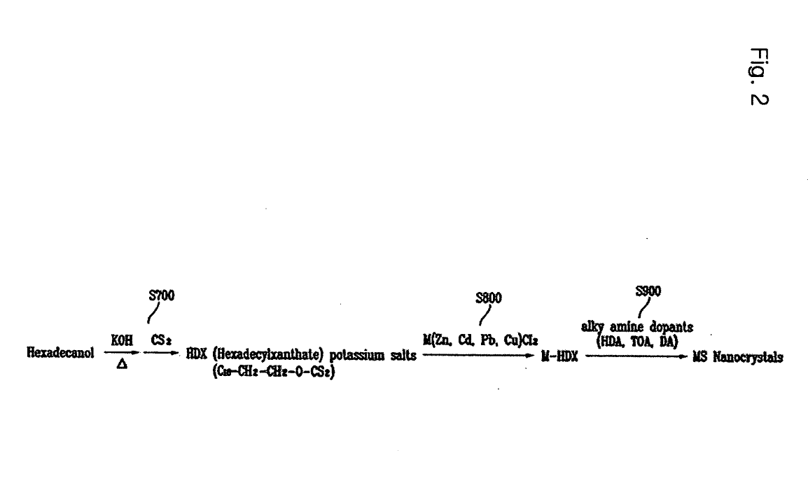

Hereinafter, a method for detecting a specific biomaterial using a nanoparticle label according to an embodiment of the present invention will be described in detail with reference to FIGS. 1a and 1b.

FIG. 1a schematically shows a method for detecting DNA using nanoparticle labels according to an embodiment of the present invention. FIG. 1a illustrates a method for performing detection using nanocrystal labels in the case where materials to be detected are four kinds of DNA fragments.

First, four kinds of DNA fragments complementary to four kinds of DNA fragments (T1, T2, T3 and T4) to be detected, respectively, were obtained, and inventive MS nanoparticle was bound to each of the obtained DNA fragments. Herein, ZnS, CdS, PbS and CuS nanoparticles were bound to the DNA fragment complementary to the DNA fragment (T1), the DNA fragment complementary to the DNA fragment (T2), the DNA fragment complementary to the DNA fragment (T3), and the DNA fragment complementary to the DNA fragment (T4), respectively.

Then, the prepared DNA fragments labeled with the inventive nanoparticles were added to samples containing four kinds of DNA fragments (T1, T2, T3 and T4) to be detected and were subjected to DNA hybridization (S100).

At this time, after removing unhybridized DNA fragments, the hybridized DNA fragments were dissolved in a nitric acid solution. Then, an electrode was placed in the nitric acid solution, and a specific negative potential was applied to the electrode, thus collecting the inventive nanoparticles having a cationic nature. If nanoparticles having an anionic nature are used as the inventive nanoparticles, positive potential can be applied to collect the nanoparticles.

The nanoparticles collected on the electrode as described above was subjected to voltammetric stripping as one of nanoparticle detection methods, thus measuring a current peak corresponding to each of the nanoparticles (S200).

Then, the measured current peak corresponding to each of the nanoparticles was sampled, converted to a digital signal and then output (S300).

FIG. 1a shows the results of using barcodes as digital signals. By analyzing such digital signals, a nanocrystal corresponding to the relevant digital signal can be identified, and a DNA fragment having the identified nanocrystal bound thereto, and a DNA fragment capable of complementarily binding to the identified fragment, can be sequentially identified. Thus, through the analysis of the digital signal, it can be seen that a DNA fragment to be detected is present in a sample to be detected, in the amount indicated by the corresponding digital signal.

FIG. 1b schematically shows a method for detecting an antigen using nanoparticle labels according to an embodiment of the present invention.

First, the inventive MS nanoparticles were bound to four kinds of antibodies (Ab1, Ab2, Ab3 and Ab4), which could specifically bind to four kinds of antigens (Ag1, Ag2, Ag3 and Ag4) to be detected, respectively. Herein, ZnS, CdS, PbS and CuS nanoparticles were bound to the antibody (Ab1), the antibody (Ab2), the antibody (Ab3) and the antibody (Ab4), respectively.

Then, the prepared antibodies labeled with the inventive nanoparticles were added to samples containing the antigens to be detected and were subjected to a sandwich immune response (S400).

At this time, after removing antibodies labeled with the inventive nanoparticles, which were not subjected to specific binding to the antigens, the bound antigen-antibody complex was dissolved in a nitric acid solution. Then, an electrode was placed in the nitric solution, in which the complex was applied with a specific negative potential, thus collecting the inventive nanoparticles having a cationic nature. The collected nanoparticles were applied with a specific stripping voltage to measure the characteristic signal peak of each of the nanoparticles (S500).

Then, the measured current peak corresponding to each of the nanoparticles was sampled, converted to a digital signal and then output (S600). By analyzing such digital signals, a nanocrystal corresponding to the relevant digital signal can be identified, and an antibody having the identified nanocrystal bound thereto, and an antigen capable of complementarily binding to the identified antibody, can be sequentially identified. Thus, through the analysis of the digital signal, it can be seen that an antigen to be detected is present in a sample to be detected, in the amount indicated by the corresponding digital signal.

Hereinafter, a method for preparing nanoparticles according to an embodiment of the present invention will be described in detail with reference to FIG. 2. The nanoparticles according to the embodiment of the present invention have high water solubility and physical chemical stability and show high biocompatibility with biomaterials.

The method for preparing the nanoparticles according to the inventive embodiment comprises a step (S700) of preparing hexadecyl xanthate (hereinafter, referred to as “HDX”) potassium salts of metal, a step (S800) of synthesizing metal sulfide nanoparticles, and a step (S900) of stabilizing and capping the surface of the nanoparticles.

FIG. 2 is a conceptual diagram schematically showing the method for preparing the nanoparticles according to the inventive embodiment.

First, the step (S700) of preparing the HDX potassium salts will be described. In this embodiment, HDX serves to stably cap the metal nanoparticles to produce metal sulfides. In FIG. 2, zinc, cadmium, lead and copper were used as metals for the nanoparticles.

9.69 g (0.04 mole) of hexadecanol was mixed with 2.24 g (0.04 mole) of potassium hydroxide (KOH) and heated at a temperature of 150° C. until the mixed solution was completely dissolved. Then, the mixed solution was uniformly stirred in 25 ml of toluene at a temperature of 100° C. Then, 4.41 g of carbon disulfide were added thereto in very small amounts at room temperature, thus obtaining a yellow solution. Following this, the yellow solution was strongly stirred for 1 hour and then additionally stirred in 100 ml of petroleum ether for 2 hours. Then, the solution was filtered through a glass funnel and washed with ether, and the filtration and washing step was repeated several times, thus obtaining HDX potassium salts as final products. Specifically, the HDX potassium salts were completely dried in a vacuum oven, washed with 20 ml of cold distilled water, filtered through a glass funnel, dried, washed with ether, washed three times with methanol, and then dried, thus obtaining the HDX potassium salts as final products.

In this embodiment, the HDX potassium salts (C₁₆CH₂CH₂OCS₂⁻), having uniform particle size and high solubility and purity, could be obtained through the multi-step filtering and washing process as described above.

Then, the step (S800) of synthesizing the metal sulfide nanoparticles and the step (S900) of stabilizing the surface of the nanoparticles will be described.

The above-obtained HDX potassium salt (C₁₆CH₂CH₂OCS₂⁻) was decomposed at low temperature. Then, 3.56 g of the HDX potassium salt was dissolved in 5 ml of methanol and allowed to react with the same molar amount of each of CdCl₂, PbCl₂, ZnCl₂ and CuCl₂ for 2 minutes. After completion of the reaction, each of the mixed solutions was centrifuged and the supernatant was removed, yielding metal HDX sulfide nanoparticles (S800). The obtained metal HDX was washed with methanol and dried in a vacuum oven.

Then, the obtained metal HDX was mixed with an alkyl amine dopant. The alkyl amine dopant is thought to have strong electron donor ability and stabilize the metal HDX single layer. Although the alkyl amine dopant used in the inventive embodiment is not specifically limited, as long as it has strong electron donor ability and can stabilize the metal HDX, it is preferable to use hexadecylamine (hereinafter, referred to as “HDA”), decylamine (hereinafter, referred to as “DA”) and/or trioctylamine (hereinafter, referred to as “TOA”). Specifically, in this embodiment, HAD was used for Zn-HDX and Cd-HDX, TOA and DA were used for Pb-HDX, and TOA and HDA were used for Cu-HDX.

Before the alkyl amine dopant was mixed with the metal HDX, it was heated to 120° C. and cooled to 50° C. Then, 0.05 g of the metal-HDX powder was added while it was uniformly stirred. Then, the mixture was stirred for 30 minutes while heating it to 100° C., after which the stirred mixture was slowly heated to 120° C. and then continued to react for 1.5 hours. After the reaction mixture was subjected to a final reaction for 140° C. for 2 minutes, the temperature thereof was slowly lowered to 70° C.

The resulting metal crystal particles were white ZnS nanocrystal particles, yellow CdS nanocrystal particles, black PbS nanocrystal particles and bluish green CuS nanocrystal particles, respectively. These nanocrystal particles were flocculated with methanol so that they were precipitated on the bottom of test tubes for easy extraction. Then, the nanoparticles were subjected to a centrifugation and supernatant removal process several times, and dried at room temperature, yielding final nanocrystal particles in the form of fine powder.

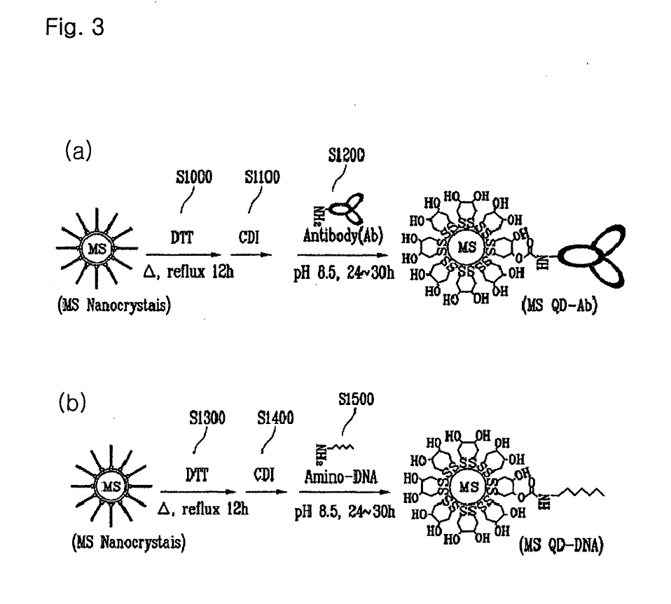

A method for preparing nanoparticle-biomaterial complexes by binding the obtained nanoparticles as labels to a given biomaterial will now be described in detail with reference to FIG. 3. In this embodiment, anti-Mb, anti-HSA, anti-β₂-MG and anti-CRP antibodies were used as biomaterials.

FIG. 3 is a conceptual diagram schematically showing a method for preparing nanoparticle-biomaterial complexes according to the inventive embodiment.

FIG. 3a conceptually shows a method for preparing nanoparticle-antibody complexes according to the inventive embodiment. FIG. 3b conceptually shows a method for preparing nanoparticle-DNA complexes according to the inventive embodiment.

The method for preparing the nanoparticle-biomaterial complexes comprises allowing the above-obtained metal nanoparticles (MS) to react with a stabilizing agent so as to be stabilized, activating the nanoparticles with an activating agent and then allowing the nanoparticles to react with a biomaterial, thus obtaining nanoparticle-biomaterial complexes.

The stabilizing agent, used in the inventive embodiment, serves to physically and chemically stabilize the nanoparticles and increase the solubility of the nanoparticles to increase the biocompatibility of the nanoparticles with the biomaterial, thus contributing the stabilization of the nanoparticle-biomaterial complexes. Specifically, the stabilizing agent consists of a polymer substance, which has a chemical group capable of binding to the nanoparticles, at one side thereof, and a plurality of water-soluble groups at the opposite side thereof. The stabilizing agent binds to the nanoparticles by surrounding the nanoparticles with the chemical groups at the one side thereof, and protects the nanoparticles from a water-soluble medium through the water-soluble groups at the opposite side thereof, thus ensuring the stability of the nanoparticles. Also, it forms covalent bonds with the biomaterial through a portion of the water-soluble groups at the opposite side, thus promoting the binding between the nanoparticles and the biomaterial.

In the inventive embodiment, dithiolthreitol (hereinafter, referred to as “DTT”) or dihydrolipoic acid (hereinafter, referred to as “DHLA”) is preferably used as the stabilizing agent. This DTT or DHLA greatly stabilizes the nanoparticles due to the surface chemical properties thereof and the high solubility thereof in aqueous solution. Specifically, DTT strongly and uniformly surrounds the nanoparticle surface at the nano-size level by a thiol group (—SH) on the molecular structure thereof, so that it maintains the stable structure of the nanoparticles and, at the same time, produces a hydroxyl group on the nanoparticle surface, resulting in an increase in the water solubility of the nanoparticles. Herein, the binding-stabilizing agent is thought to bind to the nanoparticles having a positive charge property by uniformly surrounding the nanoparticles using the negative charge property of the thiol (—SH) group. Thus, as the binding-stabilizing agent, it is preferable to use a material in which pluralities of substituent groups having a negative charge property are present at the one side thereof to uniformly surround the nanoparticles so as to stabilize the nanoparticles, and pluralities of water-soluble substituent groups are present at the opposite side thereof so as to increase the water solubility of the nanoparticles. In the inventive embodiment, the thiol (—SH) group was exemplified as the substituent group having a negative charge property, but it is thought that other substituent groups having a negative charge property can be used and, as a specific example thereof, an hydroxyl (—OH) group may also be used. Because this embodiment uses the nanoparticles having a positive charge property, a stabilizing agent having a negative charge property is preferably used, but when nanoparticles having a negative charge property are used, it is preferable to use a stabilizing agent having a substituent group bearing a positive charge property.

The activating agent, used in the inventive embodiment, serves to induce the activation of the stabilizing agent, so that the stabilizing agent can form a carbamate bond with the amino group of the biomaterial. In the inventive embodiment, 1,1-carbonyl diimidazole (hereinafter, referred to as “CDI”) was preferably used as the activating agent.

A method for preparing nanoparticle-antibody complexes will now be described with reference to FIG. 3a. As shown in FIG. 3a, nanoparticles were stirred with DTT for 12 hours so as to be hydroxylated (S1000), and then activated with CDI (S1100). The activated metal nanoparticles (CdS, PbS, ZnS and CuS) were allowed to react with 100 μl of each of anti-Mb, anti-HSA, anti-β₂-MG and ant-CRP (240 M in 20 mM phosphate buffer solution (PBS), pH 7.4) by stirring at room temperature for 24 hours (S1200). After completion of the reaction, unreacted antibodies were removed with dioxane. The resulting material was dispersed in 0.1 M PBS (pH 7.4, 0.05% Tween 20).

FIG. 3a, MS QD-Ab conceptually shows that metal sulfide quantum dot nanoparticles were bound to antibodies through DTT.

A method for preparing nanoparticle-DNA complexes will now be described with reference to FIG. 3b. As shown in FIG. 3b, nanoparticles were hydroxylated by stirring with DTT for 12 hours (S1300), and then activated with CDI (S1400). The activated metal nanoparticles (CdS, PbS, ZnS and CuS) were allowed to react with 100 μl of each of four kinds of amino-DNA by stirring at room temperature for 24 hours (S1500). After completion of the reaction, unreacted DNA fragments removed, yielding nanoparticle-DNA complexes.

In FIG. 3b, MS QD-DNA conceptually shows that metal sulfide quantum dot nanoparticles were bound to DNA through DTT.

As can be seen in FIGS. 3a and 3b, the nanoparticles according to the inventive embodiment stably bind to biomaterials, such as antibodies or DNA, through DTT activated by CDI.

Hereinafter, a detection method capable of detecting nanoparticle labels bound to biomaterials according to an embodiment of the present invention will be described.

The inventive embodiment uses an electrochemical assay as a method for detecting nanoparticle labels. The electrochemical assay, used in the present invention, is carried out in an aqueous solution to measure potential, current, electrical conductivity, impedance, capacitance, resistance or the like, and is useful in a small-scale array, because it can realize miniaturization and rapid signal processing.

In the inventive embodiment, square-wave anodic stripping voltammetry among electrochemical assays was used.

The stripping voltammetry used in the inventive embodiment broadly consists of two steps. First, biomaterials labeled with nanoparticle labels are placed in a given aqueous solution, in which an electrode is placed and a specific potential is applied to the nanoparticles through the electrode. According to the applied potential, the nanometal particles move to the direction of the relevant electrode, so that, they are collected on the relevant electrode. Then, a given potential is applied to the nanoparticle metals collected on the relevant electrode, so that a specific current flows through the nanoparticle metals. Herein, each of the nanoparticle metals generates a specific peak of electric current by oxidation and reduction reactions depending on the kind of each of the nanoparticle metals, and this specific peak of current is measured to determine the presence of the nanoparticle labels and the concentration thereof.

The method for detecting the nanoparticle labels using the stripping voltammetry according to the inventive embodiment provides picomolar levels of detection limit. This detection limit is achieved through the use of high-purity nanoparticles according to the inventive embodiment. Also, it is possible to significantly increase the sensitivity of sensor signals by catalytically controlling the size of the nanocrystals according to the inventive embodiment.

When pluralities of biomaterials are to be simultaneously detected using the nanoparticles according to the inventive embodiment, each of different metal nanoparticles is used for the respective biomaterials. In this case, each of the nanoparticles shows a specific current peak depending on the kind of metal so as to enable the pluralities of biomaterials to be simultaneously detected.

The square-wave anodic stripping voltammetry used for electrochemical detection in the inventive embodiment was carried out in Autolab 12 (Eco Chemie, Netherlands) operated with the GPES software. For analysis, a 2×4 mm size screen printing carbon work electrode (Acheson-ink), an Ag/AgCl reference electrode (CH Instruments, Austin, Tex.) and a platinum counter electrode (CH Instruments, Austin, Tex.) were used in a 1.5-ml glass cell. Specifically, as the screen printing carbon electrode, an electrode coated with mercury (II) ions or bismuth ions was used. All centrifugation processes were carried out using the Micromax centrifuge (Thermo IEC, MA).

The square-wave anodic stripping voltammetry used in the inventive embodiment will now be described in detail.

First, biomaterials labeled with the nanoparticle labels are dissolved in an aqueous nitric acid solution, and then an electrode is placed in the nitric acid solution.

Then, the biomaterials are pretreated by applying a voltage of 0.6 volt through the electrode for 1 minute and, the metal nanoparticles are collected toward the electrode by applying a negative potential of −1.4 V for 2 minutes. Herein, 1 ml of 0.1M acetate buffer (pH4.5) is used. After 5 seconds of a rest period, stripping voltage is applied to measure a current peak, which is produced from each of the metal nanoparticles and peculiar to each of the metal nanoparticles. Specifically, the application of stripping voltage is performed in a potential range of 1.2-0.12 V at a step potential of 50 mV, a magnitude of 20 mV and a frequency of 25 Hz. The correction of the baseline of the obtained curves is performed using the “moving average” mode of the GPES software. All the final results are stored through the “background subtraction” option in the software itself.

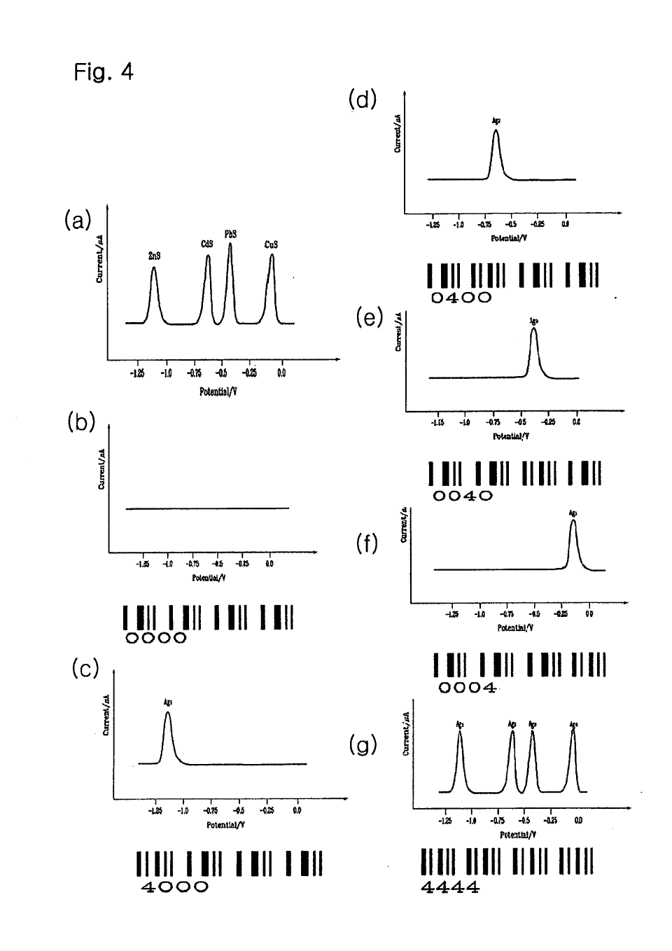

FIGS. 4a to 4g show processes for detecting four kinds of antigens using an electrochemical assay according to the inventive embodiment.

FIG. 4a shows current peaks obtained by dissolving ZnS-anti-β₂-MG, CdS-anti-Mb, PbS-anti-HSA and CuS-anti-CRP according to the inventive embodiment in nitric acid, and such current peaks in FIG. 4a are used as references for analyzing current peaks obtained by the electrochemical assay.

FIG. 4b shows barcodes obtained by converting current peak signals and the corresponding current peak signals to digital signals in the case where there is no antigen in a sample to be detected.

FIGS. 4c to 4f show barcodes obtained by converting current peak signals and relevant current peak signals to digital signals in the cases where there is one antigenic target in each of samples to be detected.

FIG. 4g shows barcodes obtained by converting current peak signals and the corresponding current peak signals to digital signals in the case where there are four kinds of antigenic targets in a sample to be detected.

Barcodes in FIGS. 4c to 4g and numerals below the bottoms show the concentrations of antigenic targets.

As can be seen in FIG. 4a, antibodies labeled with nanoparticles (ZnS, CdS, PbS and CuS), respectively, have current peaks definitively distinguished from each other, and the current peaks thereof do not change even after the antibodies have been bound to antigens (Ag1, Ag2, Ag3 and Ag4), respectively. Thus, it is possible to quantitatively detect each of the antigens contained in the sample to be detected, from the obtained current peaks. Herein, current peaks caused by each of the metal ions bound to the respective antigens were shown at voltages of −1.12 V for zinc-antigen (Ag1) (ZnS-anti-β₂-MG), −0.68V for cadmium-antigen (Ag2) (CdS-anti-Mb), −0.53V for lead-antigen (Ag3) (PbS-anti-HSA), and −0.13V for copper-antigen (Ag4) (CuS-anti-CRP).

As described above, in the anodic stripping voltammetry used in the inventive embodiment, the potential metal nanoparticles produced the current peak signals which could be selectively easily analyzed. In the inventive embodiment, metals having outstanding current peaks which do not overlap with each other in the anodic voltage range were selected as transition metals suitable for use as nanoparticles. To use the anodic stripping voltammetry, cathodic potential metals were preferred. However, in the case of using the cathodic stripping voltammetry, it is preferable to cathodic potential metals.

In the case of using the nanoparticles that produce characteristic current peaks for each electrode using both the anode and the cathode as described above, a larger amount of metals can be used as nanoparticles. As a result, it is possible to simultaneously detect a larger number of biomaterials.

The current peaks thus obtained can be digitalized according to a given digital conversion method in an analog-digital conversion unit. Specifically, the obtained analog-type current signals were sampled and output as the corresponding digital signals. The digital conversion method comprises a digitization step by the substitution of signal values, and a normalization step. The digitization is carried out by statistical optimal threshold and piecewise linear interpolation.

The resulting current peak signals are converted to digital signals depending on the magnitude and trend thereof, and can be transmitted to a remote site through wire or wireless communication means or can be stored as digital figures. Specific examples of such digital figures may include barcodes.

FIGS. 4b to 4g show barcodes converted from the current peak signals. FIG. 4b shows barcodes obtained when current peaks were not detected, and FIGS. 4c to 4f show examples in which, when nanoparticle-antibody complexes, ZnS-Ag1, CdS-Ag2, PbS-Ag3 and CuS-Ag4 were detected, the measured current peak signals of the detected complexes were converted to barcodes. FIG. 4g shows examples in which, when four kinds of nanoparticle-antibody complexes, ZnS-Ag1, CdS-Ag2, PbS-Ag3 and CuS-Ag4 were simultaneously detected, the measured current peak signals of the detected complexes were converted to barcodes. Numerals below the barcodes shown in FIGS. 4b to 4g correspond to the characteristic current peaks of Zn, Cd, Pb and Cu, respectively, and the magnitude thereof corresponds to the magnitude of the measured current peaks. Thus, from the barcodes shown in FIGS. 4b to 4g, the presence and content of the corresponding nanoparticles can be determined, and the presence and content of the antibody forming a complex with the corresponding nanoparticles can be determined therefrom.

The signals thus digitalized can be read out by a given digital reader device or barcode reader device and can be stored in a given storage medium. Specifically, when the inventive diagnostic kit is used in hospitals, diagnostic results for each of patients can be digitalized and stored in a patient sample database in the hospitals.

Furthermore, the band width of barcodes for each of marker individuals of biomaterials (protein, DNA, RNA and cells) can be integrated with wireless medical diagnostic communication devices using code division multiple access (hereinafter, referred to as “CDMA”) or ubiquitous technology, which are recently highlighted wireless IT technologies.

As described above, the electrochemical assay according to the present invention shows stable results. The stability of the electrochemical assay was evaluated by a test five times repeated. As can be seen in Table 1 below, the electrochemical assay showed a very high reproducibility in a sample containing four different antigens at a concentration of 100 ng/ml (level 4).

TABLE 4

| | Current peak | Detection | |
|---|---|---|---|
| Antigens | voltage | limit (ng/ml) | R.S.D.(%) |
| β₂-MG (Ag₁) | −1.11 | 10.6 | 9.3 |
| Mb (Ag₂) | −0.67 | 9.5 | 7.1 |
| HSA (Ag₃) | −0.45 | 9.8 | 11.2 |
| CRP (Ag₄) | −0.08 | 12.1 | 10.3 |

As can be seen in Table 1, the antigens (Ag1, Ag2, Ag3 and Ag4) bound to zinc nanoparticles, cadmium nanoparticles, lead nanoparticles and copper nanoparticles, respectively, showed relative standard deviations (hereinafter, referred to as R.S.D”) of 9.3%, 7.1%, 11.2% and 10.3%, respectively.

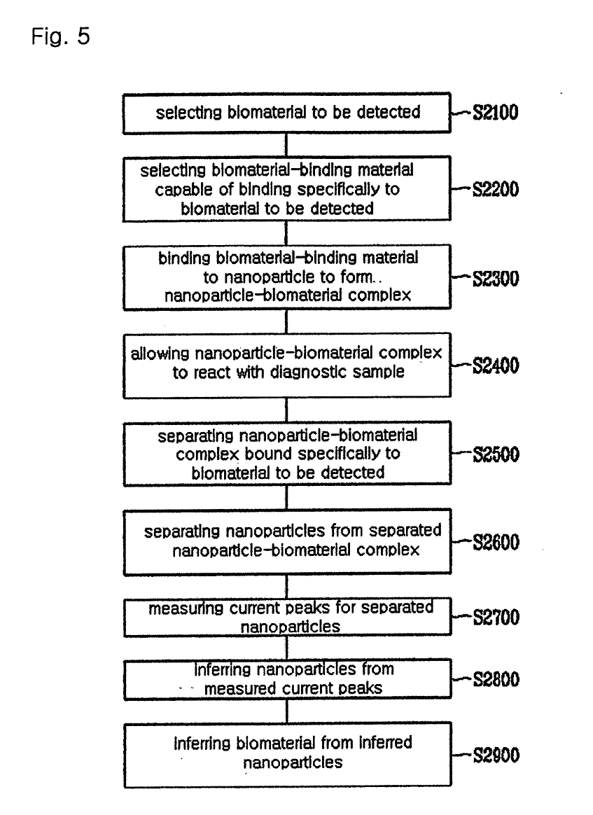

Hereinafter, a diagnostic method that uses the nanoparticle labels according to the inventive embodiment will be described with reference to FIG. 5.

FIG. 5 shows a sequential diagram of the diagnostic method that uses the nanoparticle labels according to the inventive embodiment.

As shown in FIG. 5, a biomaterial to be detected is first selected for the purpose of diagnosis (S2100). For example, when a specific disease is to be diagnosed, a specific protein caused by the corresponding specific disease is selected.

Then, a biomaterial-binding material capable of binding specifically to the biomaterial to be detected is selected (S2200). For example, when a specific antigen is to be detected, an antibody binding specifically to the corresponding specific antigen is selected as the biomaterial-binding material.

Then, the nanoparticles obtained according to the inventive embodiment are bound to the biomaterial-binding material to form a nanoparticle-biomaterial complex (S2300). Because the method for forming the nanoparticle-biomaterial complex has been described in detail above, the detailed description thereof will be omitted herein.

The formed nanoparticle-biomaterial complex is mixed with a sample to be detected, so that the binding reaction between the biomaterial to be detected and the nanoparticle-biomaterial complex is induced (S2400).

After completion of the binding reaction, the nanoparticle-biomaterial complex bound specifically to the biomaterial to be detected is isolated and identified (S2500).

The isolated nanoparticle-biomaterial complex is dissolved in an aqueous nitric acid solution, the nanoparticles are isolated (S2600), and the characteristic current peak of the nanoparticles is measured using an electrochemical assay (S2700). Because this electrochemical assay has been described above in detail, the description thereof will be omitted herein.

Then, the measured current peak corresponding to the nanoparticles is analyzed, so that the identity of the corresponding nanoparticles is inferred and (S2800), and the biomaterial bound to the nanoparticles is inferred from the inferred nanoparticles (S2900). Based on the presence or absence of the inferred biomaterial and the concentration of the biomaterial, the onset of a specific disease is diagnosed.

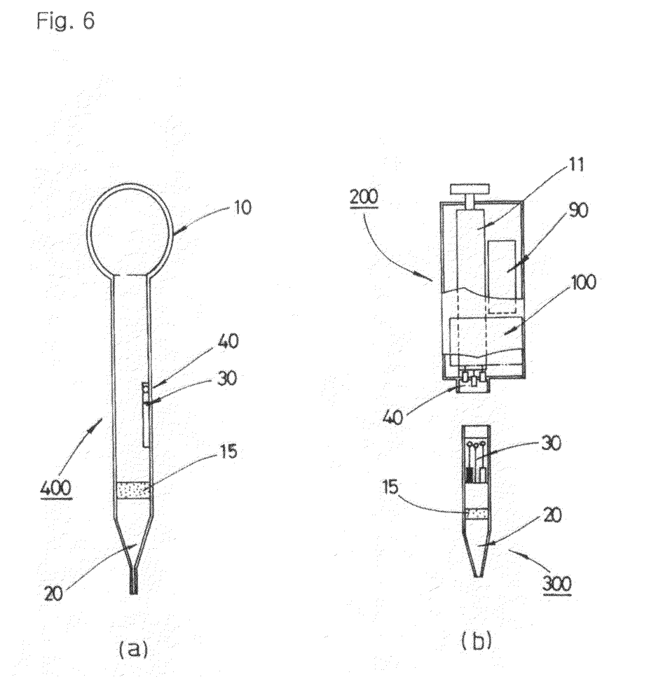

Hereinafter, a diagnostic device that uses the nanoparticle labels according to the inventive embodiment will be described with reference to FIGS. 6 to 12.

FIG. 6(a) shows a disposable dropette-type diagnostic device 400 which is connected to a rack-type docking container 500. FIG. 6(b) is a schematic diagram illustrating the main elements of a micropipette-type consisting of a disposable tip 300 and a body 200. First, the disposable dropette-type diagnostic device will be described with reference to FIG. 6(a). The diagnostic device 400 comprises a suction unit 10 for the suction of a biological sample, a sample inlet 20, a connection 40 to a rack-type docking container 500 having a potentiostat included therein, and a triode electrode 30. The disposable dropette 400 may comprise a microporous membrane for removing impurities from a biological sample. The micropipette-type diagnostic device will now be described with reference to FIG. 6(b). The micropipette-type diagnostic device comprise a disposable tip 300 including a sample inlet 20 and a triode electrode 30, and a body 200 in which a potentiostat is included, the body 200 including a pipette module 11 in which various parts such as springs and gears can be included, a connection 40 to the disposable tip 300, a mobile circuit 90 and a display module 100. The disposable tip 300 may have a microporous membrane 15 for removing impurities from the biological sample.

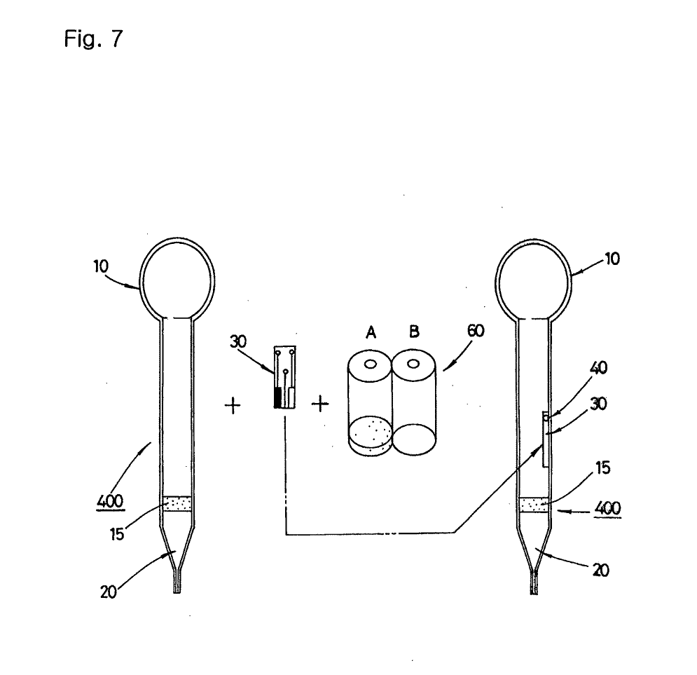

FIG. 7 shows a model of the substantial construction of the disposable dropette. The suction unit in the disposable dropette is made of an elastic material. Thus, when the suction unit is pushed down with the pressure of hand, it will push out a material from the disposable dropette, and when the pressure of hand is decreased, it will be restored to the original state using an elastic force while it will suck a material in the disposable dropette. The microporous membrane 15 serves to remove impurities from the biological sample. The triode electrode consists of screen-printed work electrodes, work electrode (W), counter electrode (C) and reference electrode (R), and is connected through the connection 40 to the rack-type docking container 500 in which a potentiostate is included. A reactor 60 consists of two parts A and B. Part A contains a reagent, containing an antibody and magnetic beads, and serves to mix the sample with the reagent, and part B is an analytic container containing a buffer solution.

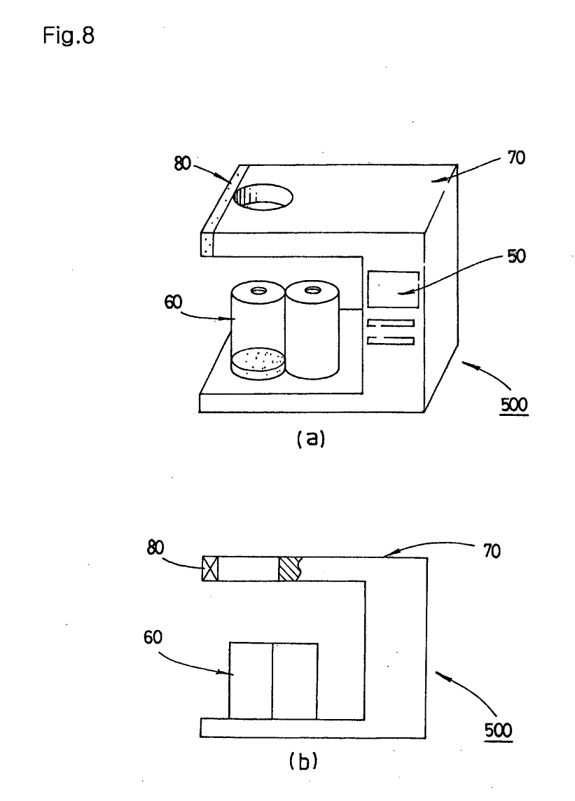

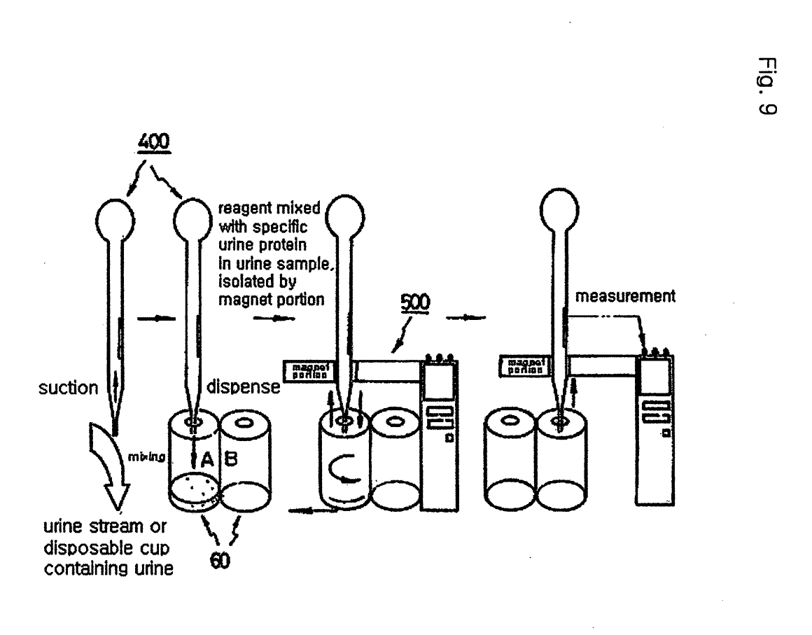

FIG. 8 shows a rack-type docking container to which the disposable dropette 200 is connected. FIG. 8(a) shows a perspective view of the rack-type docking container 60 including the reactor 60. FIG. 8(b) shows a cross-sectional view of the rack-type docking container. The rack-type docking container has a magnet part callable of selectively isolating only specific antigen-bound magnetic bead complexes using magnetic force.

FIG. 9 shows a process of immunoassaying a patient urine sample (urine protein) using a biosensor according to the present invention.

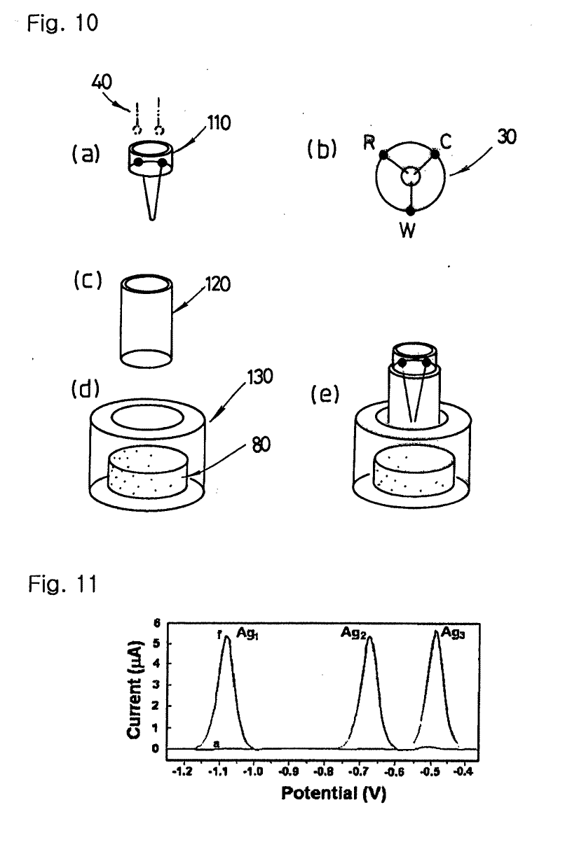

FIG. 10 is a schematic diagram of the simplest self-bioanalysis system comprising a disposable container and a stopper having an electrode attached thereto. The stopper portion 110 for the biological sample storage container in the diagnostic device has a triode electrode 30 and a connection 40 to an external potentiostat.

FIG. 11 is a graphic diagram showing anodic stripping current signals as functions of increases in the concentrations of three kinds of antigenic substances, which were simultaneously analyzed using a pipette-type sensor according to the present invention.

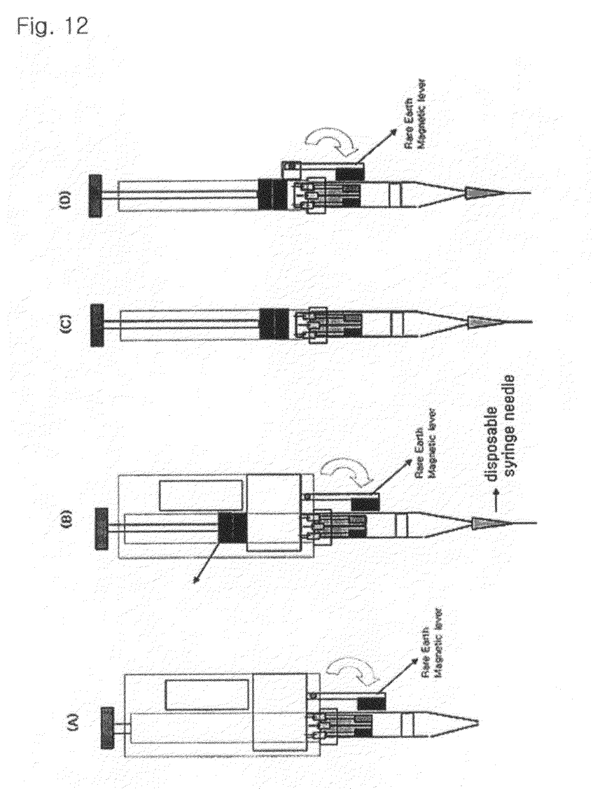

FIG. 12 is a schematic diagram showing various biosensor models in which the inventive diagnostic devices are applied. FIG. 12(A) is a model in which a micropipette and a magnetic separator are integrated with each other, and an external magnetic separator does not need to be separately designed. FIG. 12(B) is a model usable for the measurement of blood biomolecules. In the model shown in FIG. 12(B), the sample collection is performed according to the same principle as that of an existing syringe, and bioanalysis and diagnosis are completed simultaneously with the collection of blood. FIG. 12(C) shows a model in which an electrode unit is formed in a disposable syringe, and FIG. 12(D) shows a model in which the model of FIG. 12(C) is equipped with a magnetic separator lever.

### MODE FOR INVENTION

Hereinafter, a microarray immunoassay, which is one embodiment of a diagnostic method according to the present invention and is carried out using a plurality of antibodies and a nanoparticle label corresponding to each of the antibodies, will be described in detail.

First, 100 μl of PBST (phosphate buffer saline containing 0.05 (v/v) Tween 20, pH 7.2) buffer was dispensed into each well of a BD BioCoat streptavidin assay plate for about 15 minutes until the plate reached equilibrium. Then, each of the wells was washed with 100 a of TTL buffer (100 mM TrisHCl, pH 8.0, 0.1% Tween and 1 M LiCl). Each of previously obtained nanoparticle-labeled antibodies was diluted to a concentration of 1000 mg/l, and 4 μl of each dilution was mixed with 84 μl of TTL buffer and incubated with shaking (100 rpm) at room temperature for 30 minutes. Then, the supernatant was removed by aspiration, and each well was washed two times with 100 μl of TTL buffer (250 mM TrisHCl, 0.1% Tween 20).

Then, 5 μl of each of four kinds of antigens having different antigen concentrations was added to 80 μl of TTL buffer (750 mM NaCl, 150 mM sodium citrate), was dispensed into microwells having capture antibodies fixed thereto, and then was incubated for 30 minutes, so that each of the antigens was allowed to react with the corresponding capture antibody. Then, each of the wells was washed with 100 μl TTL buffer.

Then, the nanoparticle-labeled antibodies pretreated as described above were dissolved in 100 μl of TTL buffer, and the nanoparticle-labeled solution was added to the wells captured with the antigens, and were incubated for 30 minutes to perform antigen-antibody reactions. Then, each of the well was washed with 100 μl of TTL buffer.

Table 2 below shows capture antibodies (anti-β₂-MG, anti-Mb, anti-HAS and anti-CRP) and nanoparticle-labeled antibodies (ZnS-anti-β₂-MG, CdS-anti-Mb, PbS-anti-HSA, and CuS-anti-CRP), which correspond to the used antigens (β₂-MG, Mb, HSA, and CRP), respectively. In this respect, the capture antibodies (anti-β₂-MG, anti-Mb, anti-HSA, and anti-CRP) were biotinylated.

TABLE 2

| | | Nanoparticle-antibody |
|---|---|---|
| Antibodies | Capture antibodies | complexes |
| β₂-MG (Ag₁) | Anti-β₂-MG | ZnS-anti-β₂-MG |
| Mb (Ag₂) | Anti-Mb | CdS-anti-Mb |
| HSA (Ag₃) | anti-HSA | PbS-anti-HSA |
| CRP (Ag₄) | anti-CRP | CuS-anti-CRP |

Then, each of the complexes was stirred in 20 u of 1 M nitric acid aqueous solution for 3 minutes, so that the nanoparticle-antibodies bound to the well. Then, 1 ml of acetate buffer (0.1 M, pH 4.5) containing 10 ppm of a mercury atom absorbance reference solution, which could measure all the four metals used in this embodiment, was added to the dissolved nanoparticle label, and the characteristic current peak of each of the metal nanoparticles was measured using the above-described electrochemical assay.

As can be seen in Table 1 above, the detection limit of ZnS-anti-β₂-MG Was 10.6 ng/ml, the detection limit of CdS-anti-Mb was 9.5 ng/ml, the detection limit of PbS-anti-HAS was 9.8 ng/ml, and the detection limit of CuS-anti-CRP was 12.1 ng/ml, suggesting that the electrochemical assay according to the inventive embodiment was very low.

Thus, when the concentration range (40-120 mg/l) of proteins detected from the urine of a typical Proteinuria patient is considered as a range indicating the dangerous level in diagnosis, the use of the inventive nanoparticle labels enables even a very low concentration of disease factors to be detected, because the detection limit of the nanoparticle labels is much lower than said concentration range.

This sensor performance is also shown at almost the same level as described above, when DNA is used as an analytical material. Specifically, when the nanoparticle labels according to the inventive embodiment are bound to probes capable of detecting cancer genes causing bladder cancer, breast cancer and the like, and then are used for diagnosis, the cancer genes can be diagnosed at a high level.

Meanwhile, for higher sensitivity, bimetallic nanoparticles (e.g., CdS/ZnS core/shell structure) having a slightly increased size and shape can be used. The bimetallic nanoparticles are obtained by binding two kinds of nanoparticles to each other. Specifically, an alloy nanoparticle structure having increased size is formed by applying a shell of one kind of nanoparticles to a core of another kind of nanoparticles. The bimetallic nanoparticles include CdS/ZnS, CdS/Pbs and CuS/ZnS, all of which form a core/shell structure.

As described above, the present invention provides a method for quantitatively analyzing a biomaterial to be detected, using a nanoparticle label as a signaling label. By quantitatively detecting a specific biomaterial such as an antibody or DNA in a specific sample as described above, it is possible to diagnose the presence or absence of a specific disease in a subject.

Thus, it is possible to construct a kit for diagnosing a specific disease, comprising the inventive nanoparticle label, a means for measuring nanoparticles, and a means for converting measurement results to digital signals. When digital means such as barcodes are used as described above, the diagnosis of disease can be more easily performed.

As used in the inventive diagnostic kit, the term “nanoparticle labels” refers to nanoparticles bound to another biomaterial for detecting a specific biomaterial. Specifically, the term refers to nanoparticles bound to a DNA fragment having a DNA sequence complementary to that of a specific DNA fragment, in order to detect the specific DNA fragment. As another example, the term refers to nanoparticles bound to a specific antibody binding specifically to a specific antigen, in order to detect the specific antigen.

These nanoparticle labels in the inventive diagnostic kit can be replaced depending on the kind of a biomaterial to be detected. Thus, the medical/biological diagnosis of various substances, including DNA and RNA molecules (prediction of mutations by base sequencing), peptides, cancer markers, drugs (narcotics), microorganisms (0157 bacteria, food poisoning bacteria, and Treponema pallidum ko, can be achieved in one diagnostic kit only by replacement of the nanoparticles labels.

The means for measuring the nanoparticles in the inventive diagnostic kit is not specifically limited as long as it can realize the electrochemical assay as described above.

Also, the means for converting measurement results to digital signals may be a means for outputting barcodes as described above, but is not limited thereto and can be integrated with up-to-date wireless mobile IT technologies, including GSM (global system for mobile), Bluetooth, Ubiquitous, and CDMA (code division multiple access).

When diagnostic results for a specific sample are output as barcodes as described above, the user of the diagnostic kit can rapidly understand the diagnostic results by reading the output barcodes with a barcode reader disposed in hospitals and the like. When the diagnostic results are output as digital signals as described above, the diagnostic results can be transmitted to a remote site through wire or wireless communication. Thus, the remote site having a system capable of evaluating the diagnostic results can receive the diagnostic results output from the diagnostic kit, evaluate the diagnostic results, and transmit the evaluation result to the user of the diagnostic kit. Thus, the inventive diagnostic kit can be used not only in medical and clinical applications, but also in surveillance systems for the environmental monitoring fields of water, food and the like and for the fields of biological warfare, TNT, crimes (narcotics) and the like, the fields employing various probes.

As described above, the nanoparticle labels according to the inventive embodiment are conveniently synthesized, and each of the metal nanoparticles has and shows a characteristic oxidation reduction potential, so that the pluralities of the nanoparticles can be simultaneously detected. Thus, when the nanoparticle labels according to the inventive embodiment are used, it is possible to detect a number of biomaterials and to miniaturize diagnostic kits.

The inventive biosensor was fabricated in the following manner.

As shown in FIG. 6(a), a screen-printed triode electrode was inserted into a suitable location in a disposable dropette widely used in the prior art. An external potentiostate was connected to the disposable dropette, thus fabricating a biosensor in which a urine protein in a sample container could be quantitatively analyzed. The biosensor could selectively isolate only specific antigen-bound magnetic bead composites using magnetic force, and thus could complete an immunoassay process only by a step of injecting and sucking fluid with a pipette. Herein, the dropette is made of low-density transparent polyethylene and has a one-pieced structure in which a bulb is combined with a pipette. The surface material having low affinity prevents noise caused by the binding between proteins and foreign matter and allows a fixed sucking and dropping size of about 25 mL.

As shown in FIG. 6(b), according to the present invention, a biosensor was fabricated by placing a potentiostat module in a micropipette widely used in the prior art and inserting an electrode into a disposable tip. The biosensor enabled a patient's sample to be more easily quantitatively analyzed and observed. In this case, a mobile module was placed in the pipette.

Also, according to the present invention, a disposable triode electrode was inserted into a stopper for a prior disposable glass tube container, and the glass container containing a reagent and a sample was covered with the stopper. Then, the glass container was attached to a magnetic platform. According to this simple process, diagnosis was completed.

Also, as shown in FIG. 11, the voltage signature of metal nanoparticles was examined in the following manner. In the present invention, in order to obtain a non-overlapping pool of semiconductor nanoparticle tracers considering a high selectivity of a bioassay, ZnS, PbS and CdS nanoparticles were selected and introduced. The current peaks of the metal ions for antigens were observed at voltages of −1.12 V (Zn), −0.68 V (Cd) and −0.53 V (Pb) (see FIG. 11).

Also, an electrochemical multiple immunoassay that uses the inventive biosensor was carried out in the following manner. First, according to square-wave anodic stripping voltammetry (SWASV), a 2×4 mm screen-printed screen-printed carbon (Acheson-ink) working electrode, an Ag/AgCl reference electrode (CH Instruments, Austin, Tex.) and a platinum counter electrode were used in a 1.5 ml glass cell. The square-wave anodic stripping voltammetry (SWASV) was carried out using a screen-printed carbon paste electrode coated with bismuth ions.

As shown in FIG. 1, in container A, QD-antigen nanocomplexes were pretreated at 0.6 V for 1 minute and then accumulated at −1.4 V for 1 minute. At this time, as buffer, 1 ml of acetate buffer in container B (pH 4.5) of FIG. 1 was used, and stripping was carried out after 5 second of a rest period without stirring. In this process, the following device operation parameters were used: a potential range of −1.2 V to 0.12 V; a step potential of 50 mV; an amplitude of 20 mV; and a frequency of 25 Hz.

Regarding the biosensor performance, in order to examine an antigen-antibody cross reactions as negative signals, non-specific antigens, hemoglobin (Hb) and bovine serum albumin (BSA) were added to a sample to measure signals, and thus almost negligible signals were detected in the sample. These signals were almost similar to noise signals in a sample free of an antigen. Accordingly, it could be observed that the inventive biosensor had high sensitivity and selectivity of effectively removing nonspecific signals, and thus showed high efficiency in actual diagnosis. In this case, potentials detected in the sample were −1.11 V (b2-MG), −0.67 V (Mb), and −0.45 V (HSA), and stable signals were shown (see FIG. 11).

The nanocrystal particles were observed for the size and shape thereof through a TEM electron microscope and, as a result, the CdS, ZnS and PbS nanocrystal particles showed sizes of about 3.9 nm, 4.5 nm and 15.7 nm, respectively. The magnitudes and locations of the final voltage peaks were consistent with the concentrations of the antigens, suggesting that a multi-target quantitative assay could be easily performed. In other words, an increase in the concentration level in kidney and cardiovascular diseases could be predicted as the increase of antigens as shown in FIG. 11 (25-125 ng/ml). FIG. 11 is a graphic diagram showing anodic stripping current signals as a function of increases (25 ng/mL) in the concentrations of three kinds of antigenic substances, which were simultaneously analyzed using the inventive pipette-type sensor. A flat baseline between peak voltage ranges (range between Cd and Zn peaks or range following Pb peak) suggests that 6-8 kinds of antigens can be simultaneously analyzed (see FIG. 11). For analysis of this increased number of antigens, Ga, Cu, As, Tl or Bi metal particles can be used.

Also, the stability of the sensor was evaluated through a test repeated five times. As a result, the sensor showed a very high reproducibility in a sample containing three different antigens at a concentration of 100 ng/ml (level 4). As shown in Table 3 below, Zn, Cd and Pb showed standard deviation peaks of 9.3%, 7.1%, and 11.2%, respectively.

TABLE 3

| Substances | QD potential | Detection | |
|---|---|---|---|
| analyzed | (V) | limit | R.S.D.(%) |
| β₂-MG(Ag1) | −1.11 | 10.6 | 9.3 |
| Mb(Ag2) | −0.67 | 9.5 | 7.1 |
| HSA(Ag3) | −0.45 | 9.8 | 11.2 |

When a protein concentration range (40-120 mg/L) detected in the urine of a typical proteinuria patient is considered as a range indicating a dangerous level in diagnosis, the inventive sensor showed a detection limit of 10.5 ng/ml in 25 ng/ml of an antigenic sample. This result suggests that the inventive sensor can detect a very low concentration of disease factors even at a concentration range much lower that the dangerous level of patients. Also, as shown in FIG. 11, this can be demonstrated from signals proportional to increases in the concentrations of antigens. Also, this low detection limit shows very increased sensitivity and selective resolution compared to those of optical immunosensors reported in the prior literatures. For the amplification of higher sensitivity, bimetallic nanoparticles (e.g., CdS/ZnS core/shell structure) having controlled size and shape can be used as a good candidate in the future.

Using the inventive biosensor, signal processing in barcode, RFID and ubiquitous systems was carried out in the following manner. The acquired analog signals were digitalized by digital signal processing, and barcode, RFID and ubiquitous systems were used for medical diagnosis and communication. In multiplex electric protein coding, the multi-redox coding of an immunoassay was performed for three different QD particles at precisely controlled concentrations. The electrical tenability of such particles contributed to the optimization of the electrochemical multiplex method.

Digital signal processing for making barcode readout rapid was performed by a digital program for digitalizing the obtained linear analog coding signals to barcodes. The allotted flexible band widths were digitalized by physical sensor modules inside and outside the sensor, and an actual patient's sample was confirmed through a database. Also, the bandwidths of DNA, RNA, cells and proteins can be divided using the CDMA technology widely used in the mobile phone technology.

Although the preferred embodiment of the present invention has been described for illustrative purposes, those skilled in the art will appreciate that various modifications, additions and substitutions are possible, without departing from the scope and spirit of the invention as disclosed in the accompanying claims.

### INDUSTRIAL APPLICABILITY

As described above, the present invention provides a medical digital processing-based point-of-care device for performing the multiplex analysis of biomaterials using a convenient-to-use small-size electrochemical sensor. This system is a biosensor which can be used for the diagnosis of a wide spectrum of substances, including proteins, DNA, virus and bacteria, through replacement of only probes. At the same time, it is a convenient diagnostic device enabling analysis time and processes to be significantly reduced. Also, it satisfies all requirements for small-scale diagnostic systems, including hand-held, battery-powered, real-time and easy-to-use properties, which are the advanced essential properties of electrochemical measurement systems, which greatly reduce shortcomings with other devices while having significant sensitivity.

Also, a medical signal communication system by barcode wireless remote communication, which is the key terchnology of the inventive biosensor, has an important effect in that it can early monitor the real-time condition of patients at the molecular level and enables the monitored results to be used as information. Also, it enables clinical information on patient's diseases to be rapidly interchanged, and thus effectively reduces many shortcomings which have been required in prior hospital diagnosis. Furthermore, through only replacement of sensor probes for various biomolecues, including DNA, proteins, peptides and hormonal receptors, the inventive biosensor is suitable for the medical/biological detection of various analytes, including proteins, DNA and RNA molecules, peptides and microorganisms. Also, it is expected to be introduced in the environmental field for the examination of pollutants, water and the like, and it is expected to be widely used in the food industry for evaluating the quality of food and examining the toxicity of food. In addition, it is expected to be widely applied for the surveillance of generation of biochemical warfare, and the surveillance of terrors and crimes in military and police departments, as well as in early warning systems.

1. A nanoparticle-biomaterial complex comprising:
one or more nanoparticles selected from a metal group consisting of zinc, cadmium, lead, copper, gallium, arsenic, thallium, nickel, manganese and bismuth;
a specific biomaterial; and
a binding-stabilizing agent containing a polymer chain, which has at one side thereof a substituent group having a charge property capable of binding the stabilizing agent to the nanoparticles, and has at the opposite site a plurality of water-soluble substituent groups, whereby the binding-stabilizing agent binds to the nanoparticles through the substituent on the one side, stabilizes the nanoparticles through the plurality of water-soluble substituent groups and forms bonds with the biomaterial through the plurality of water-soluble substituent groups.

2. The nanoparticle-biomaterial complex of claim 1, wherein the nanoparticles are in the form of metal sulfides.

3. The nanoparticle-biomaterial complex of claim 1, wherein the nanoparticles are made of one or more selected from a metal group consisting of zinc, cadmium, lead and copper.

4. The nanoparticle-biomaterial complex of claim 1, wherein the nanoparticles are nanoparticle complexes formed by binding two or more nanoparticles to each other.

5. The nanoparticle-biomaterial complex of claim 1, wherein the biomaterial is selected from the group consisting of nucleic acids, including DNA or RNA, amino acid, nucleic acid-amino acid complexes, fats, glycoprotein, signaling substances, including Ca²⁺, cAMP, cGMP, IP₃ and DAG, and antibodies.

6. The nanoparticle-biomaterial complex of claim 1, wherein the binding-stabilizing agent is dithiolthreitol or dihydrolipoic acid.

7. The nanoparticle-biomaterial complex of claim 1, wherein the binding-stabilizing agent is activated by an activating substance, and the activated binding-stabilizing substance forms a bond with the biomaterial.

8. The nanoparticle-biomaterial complex of claim 7, wherein the activating agent is 1,1-carbonyl diimidazole.

9. The nanoparticle-biomaterial complex of claim 7, wherein the bond between the binding-stabilizing agent and the biomaterial is a carbamate bond.

10. A method for preparing nanoparticles for labeling a biomaterial, the method comprising the steps of:
allowing hexadecanol, potassium hydroxide and carbon disulfide to react with each other to prepare a hexadecyl xanthate (HDX) potassium salt;
allowing the obtained HDX potassium salt to react with one or more nanoparticles selected from a metal group consisting of zinc, cadmium, lead, copper, gallium, arsenic, thallium, nickel, manganese and bismuth so as to prepare HDX metal sulfide nanoparticles; and
allowing the HDX metal sulfide nanoparticles to react with an alkyl amine dopant to prepare metal sulfide nanoparticles.

11. The method of claim 10, wherein the nanoparticles are made of one or more selected from a metal group consisting of zinc, cadmium, lead and copper.

12. The method of claim 10, wherein the step of preparing the HDX potassium salt comprises the steps of:
mixing the hexadecanol with the potassium hydroxide and heating the mixed solution until it is completely dissolved;
uniformly stirring the mixed solution in toluene and adding the carbon disulfide to the stirred solution;
additionally stirring the mixed solution in petroleum ether; and
filtering the mixed solution through a glass funnel and washing the filtrate with ether.

13. The method of claim 10, wherein the alkyl amine dopant is one or more selected from the group consisting of hexadecyl amine, decyl amine and trioctyl amine.

14. The method of claim 13, wherein, for HDX zinc sulfide nanoparticles, hexadecyl amine is used as the alkyl amine dopant, and for HDX lead sulfide nanoparticles, decyl amine or trioctyl amine is used as the alkyl amine dopant, and for HDX copper sulfide nanoparticles, hexadecyl amine or trioctyl amine is used as the alkyl amine dopant.

15. A diagnostic kit for detecting a specific biomaterial using a nanoparticle label, the diagnostic kit comprising:
a nanoparticle-biomaterial complex comprising one or more nanoparticles selected from a metal group consisting of zinc, cadmium, lead, copper, gallium, arsenic, thallium, nickel, manganese and bismuth, one or more bio-binding agents binding to the nanoparticles through a binding-stabilizing agent and binding specifically to a biomaterial to be detected, and a binding-stabilizing agent forming bonds between the nanoparticles and the bio-binding agents;
an extraction solution for isolating and extracting the nanoparticles from the nanoparticle-biomaterial complex;
a collection electrode for collecting the nanoparticles from the extraction solution; and
a current peak measurement unit for measuring current peaks corresponding to the nanoparticles collected from the collection electrode.

16. The diagnostic kit of claim 15, wherein the nanoparticles are in the form of metal sulfides.

17. The diagnostic kit of claim 15, wherein the nanoparticles are made of one or more metals selected from a metal group consisting of zinc, cadmium, lead and copper.

18. The diagnostic kit of claim 15, wherein the nanoparticles are nanoparticle complexes formed by binding two or more nanoparticles to each other.

19. The diagnostic kit of claim 15, wherein the biomaterial is selected from the group consisting of nucleic acids, including DNA or RNA, amino acid, nucleic acid-amino acid complexes, fats, glycoprotein, signaling substances, including Ca²⁺, cAMP, cGMP, IP₃ and DAG, and antibodies.

20. The diagnostic kit of claim 15, wherein the binding-stabilizing agent is dithiolthreitol or dihydrolipoic acid.

21. The diagnostic kit of claim 15, which additionally comprises an analog-digital conversion unit for converting current peaks measured from the collected nanoparticles into digital signals.

22. The diagnostic kit of claim 15, which comprises four or more nanoparticle-biomaterial complexes in order to simultaneously detect four or more biomaterials.

23. The diagnostic kit of claim 15, wherein the extraction solution includes a nitric acid solution.

24. The diagnostic kit of claim 15, wherein, if the nanoparticles have a cationic property, a negative potential is applied, and if the nanoparticles have an anionic property, a positive potential is applied to the collection electrode.

25. The diagnostic kit of claim 15, wherein the current peak measurement unit serves to apply a given potential to the nanoparticles collected on the collection electrode to subject the nanoparticles to oxidation/reduction reaction, and measure the characteristic current peak of each of the nanoparticles, which is generated from the oxidation/reduction reaction of the nanoparticles.

26. The diagnostic kit of claim 25, wherein a negative or positive potential is applied to the collection electrode to collect cationic nanoparticles or anionic nanoparticles, and the current peak measurement unit measures the characteristic current peak of each of the nanoparticles, which are generated from each of the collected nanoparticles.

27. The diagnostic kit of claim 15, which additionally comprises a digital signal reader unit of analyzing the digital signal to infer the identity of a biomaterial corresponding to the digital signal and/or the content of the detected biomaterial.

28. The diagnostic kit of claim 15, which additionally comprises a barcode conversion unit for converting the digital signals to barcodes.

29. The diagnostic kit of claim 15, which additionally comprises a communication unit of transmitting the digital signal to a remote diagnostic unit through wire or wireless communication and receiving the analyzed results of the digital signal from the remote diagnostic unit.

30. A diagnostic method that uses a nanoparticle label, the method comprising the steps of:
determining one or more biomaterial-binding materials capable of binding specifically to one or more biomaterials to be detected;
selecting one or more nanoparticles from the group consisting of zinc, cadmium, lead, copper, gallium, arsenic, thallium, nickel, manganese and bismuth, and binding the selected nanoparticles to the biomaterial-binding materials, respectively, to form one or more nanoparticle-biomaterial complexes;
mixing the nanoparticle-biomaterial complexes with a sample to be diagnosed, so as to induce the binding between the biomaterials to be detected and the nanoparticle-biomaterial complexes;
separating the nanoparticle-biomaterial complexes bound to the biomaterials;
separating the nanoparticles from the separated nanoparticle-biomaterial complexes and collecting the separated nanoparticles; and
measuring characteristic current peaks corresponding to the collected nanoparticles.

31. The diagnostic method of claim 30, wherein the nanoparticles are in the form of metal sulfides.

32. The diagnostic method of claim 30, wherein the nanoparticles are made of one or more metals selected from a metal group consisting of zinc, cadmium, lead and copper.

33. The diagnostic method of claim 30, wherein the nanoparticles are nanoparticle complexes formed by binding two or more nanoparticles to each other.

34. The diagnostic method of claim 30, wherein the biomaterials are selected from the group consisting of nucleic acids, including DNA or RNA, amino acid, nucleic acid-amino acid complexes, fats, glycoprotein, signaling substances, including Ca²⁺, cAMP, cGMP, IP₃ and DAG, and antibodies.

35. The diagnostic method of claim 30, wherein the step of forming the nanoparticle-biomaterial complexes comprises the steps of:
binding to said nanoparticles a binding-stabilizing agent so as to stabilize the nanoparticles, the binding-stabilizing agent comprising a polymer chain having at one side thereof a substituent group, which has a substituent group and can bind to the nanoparticles, the polymer chain having a plurality of water-soluble substituent groups at the opposite side;
activating the binding-stabilizing agent bound to the stabilized nanoparticles; and
binding the activated binding-stabilizing agent to the biomaterial-binding material.

36. The diagnostic method of claim 35, wherein the activating step comprises allowing carbonyl diimidazole to react with the nanoparticle-binding stabilizing agent complexes so as to activate the binding-stabilizing agent.

37. The diagnostic method of claim 30, wherein, if the biomaterials to be detected is DNA or RNA, the biomaterial-binding materials are DNA or RNA including complementary chains capable of binding to the corresponding DNA or RNA.

38. The diagnostic method of claim 30, wherein, if the biomaterials to be detected are antigens, the biomaterial-binding materials are antibodies binding specifically to the antigens.

39. The diagnostic method of claim 30, which additionally comprises a step of converting the measured current peaks to digital signals.

40. The diagnostic method of claim 39, which additionally comprises the steps of:
transmitting the converted digital signals to a remote diagnostic unit through wire or wireless communication; and
receiving diagnostic results for the digital signals from the remote diagnostic unit.

41. The diagnostic kit of any one of claims 15 to 29, which further comprises a micropipette comprising a device for sucking the biomaterial sample.

42. The diagnostic kit of claim 41, wherein the micropipette comprises a disposable tip including a screen-printed disposable electrode, and an external potentiostat connected with the electrode of the disposable tip.

43. The diagnostic kit of claim 41, wherein the micropipette comprises a microporous membrane for removing impurities from the biological sample.

44. The diagnostic kit of claim 41, wherein a magnet is included in a rack-type docking container to be inserted with a reagent-containing container.

45. The diagnostic kit of claim 41, wherein the micropipette has a mobile chip included therein.

46. The diagnostic kit of claim 41, wherein an electrochemical measurement module or an optical measurement module are included in the micropipette.

47. The diagnostic kit of any one of claims 15 to 29, wherein an electrode is included in a stopper of a container for storing the biological sample.

48. A disposable tip comprising an electrode which is connected with a potentiostat.

49. The disposable tip of claim 48, which comprises a microporous membrane for removing impurities from a biological sample.
